(19) United States
(12) Patent Application Publication
(10) Pub. No.: US 2025/0331801 A1
(43) Pub. Date: Oct. 30, 2025

(54) DENOISING IN X-RAY IMAGING

(71) Applicant: Siemens Healthineers AG, Forchheim (DE)

(72) Inventor: Sai Gokul Hariharan, Forchheim (DE)

(21) Appl. No.: 19/195,508

(22) Filed: Apr. 30, 2025

(30) Foreign Application Priority Data
Apr. 30, 2024 (EP) 24173342

(51) Int. Cl. A61B 6/00; A61B 6/46; G06T 5/70

(52) U.S. Cl. CPC A61B 6/5258; A61B 6/461; A61B 6/5205; A61B 6/5294; G06T 5/70; G06T 2207/10116

(57) ABSTRACT

For denoising in X-ray imaging, at least two noise level parameters of an X-ray imaging system for at least two frequency bands are received. Each of the at least two noise level parameters is associated with one of the at least two frequency bands. A first X-ray image generated by the X-ray imaging system is received. A first denoised X-ray image is generated by applying a denoising algorithm depending on the at least two noise level parameters to the first X-ray image.

This application claims the benefit of European Patent Application No. EP 24173342, filed on Apr. 30, 2024, which is hereby incorporated by reference in its entirety.

### BACKGROUND

The present embodiments are directed to denoising in X-ray imaging and to corresponding supervised training of a denoising algorithm for denoising in X-ray imaging.

Complex X-ray guided medical procedures may expose the patient and, especially over time, the involved clinical staff to a non-negligible amount of radiation dose. In order to reduce the dose and the risk of potentially correlated health consequences, the dose should be optimized. This provides that the applied radiation dose may be as low as reasonably achievable (“ALARA”), while the necessary image quality should be maintained. However, lowering the dose during X-ray imaging results in an increase of noise and thus, a reduction in the signal-to-noise-ratio, SNR, and the image quality. Therefore, it becomes necessary to apply post processing algorithms (e.g., denoising techniques) in the imaging chain.

Known denoising algorithms deliver very good results at various dose levels. However, at very low detector entrance dose levels and low SNR levels, known denoising algorithms cannot compensate for information loss. In other words, the approach removes noise and cannot bring back the information that has not been present in the raw data due to low SNR. In addition, known approaches do not take into consideration the noise characteristics associated with different X-ray spectra.

In the publication S. Hariharan et al.: “Learning-based X-ray Image Denoising Utilizing Model-based Image Simulations,” in Shen, D., et al.: “Medical Image Computing and Computer Assisted Intervention—MICCAI 2019,” MICCAI 2019, Lecture Notes in Computer Science, vol. 11769, Springer, Cham, the generalized Anscombe transform is applied to medical X-ray imaging. Further, it is described how a trained artificial neural network may be used as a denoising algorithm based on accordingly transformed X-ray images.

The transformation of the X-ray quanta received by an X-ray detector (e.g., an indirect-detection, flat-panel detector) into a pixel gray value may be described by a succession of stages. Each stage may, for example, involve a quantum gain or spatial spreading, also denoted as blurring. It may be assumed that this process follows a linear model. Thus, an observed noise-corrupted gray value y at row r and column c of a detector array of the X-ray detector may be represented as

$$y [ r , c ] = β \cdot ( x * {k}_{q} ) [ r , c ] + g + n$$

where x represents the charges (e.g., corrupted by quantum noise) at the photo-detectors, which convolved with the stochastic spreading function k_{g}. The variable n represents electronic noise with a standard deviation, σₙ sampled at row position r and column position c, respectively. The overall scale factor is given by β. The mixed noise variance due to quantum noise and electronic noise of a detector pixel's gray value may be expressed as

$${σ}_{y}^{2} = α \cdot \overline{y} + {σ}_{n}^{2} .$$

This may be interpreted as a line with slope α and y-intercept σₙ². The parameter y̅ denotes the mean (e.g., noise-free) value of y, and the parameter a depends on imaging parameters of the X-ray imaging system that affect the X-ray spectrum, and, for example, a gain factor of the X-ray detector.

For example, α is affected, for example, by the X-ray spectrum, the imaged object, a possible spectral X-ray filter, a possible anti-scatter grid, the imaging geometry, and, for example, an operating mode of the X-ray imaging system. This makes it difficult to predict the parameters of the noise model just based on the imaging parameters. The parameters a and σₙ² may, for example, be computed directly from an X-ray image, as described, for example, in the publication S. Hariharan et al.: “Data-driven estimation of noise variance stabilization parameters for low-dose x-ray images,” Phys Med Biol., 2020, 24, 65(22), 225027.

The parameters α and σₙ² may also be obtained from the system specifications of the X-ray imaging system and calibration measurements. Once known, the parameters may be taken into account to perform a noise variance stabilization based on a variance stabilizing transformation, such as the generalized Anscombe transform, GAT,

$${y}^{′} = t ( y , α , {σ}_{n} ) = \frac{2}{α} \sqrt{α \cdot y + \frac{3}{8} {α}^{2} + {σ}_{n}^{2}} .$$

The GAT makes the noise variance independent of the signal. In fact, the noise variance is stabilized to 1. In addition to the signal dependent noise variance, however, the noise power spectrum also depends on the X-ray spectrum. Unfortunately, the GAT stabilizes only the noise variance and not the noise power spectrum.

In the publication by O. Ronneberger et al.: “U-Net: Convolutional Networks for Biomedical Image Segmentation,” (arXiv: 1505.04597), the U-Net architecture is described, a widely used CNN architecture for image segmentation, which may, however, also be used for other computer vision tasks.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the quality of denoising in X-ray imaging for different X-ray spectra is improved.

The present embodiments are based on the idea to apply a denoising algorithm depending on the at least two noise level parameters of an X-ray imaging system for at least two frequency bands, where each noise level parameter is associated with one of the at least two frequency bands.

According to an aspect of the present embodiments, a computer-implemented method for denoising in X-ray imaging is provided. Therein, at least two noise level parameters of an X-ray imaging system for at least two frequency bands are received, where each noise level parameter of the at least two noise level parameters is associated with one (e.g., exactly one) of the at least two frequency bands. A first X-ray image, which is or has been generated by the X-ray imaging system, is received. A first denoised X-ray image is generated by applying a denoising algorithm depending on the at least two noise level parameters to the first X-ray image.

Unless stated otherwise, all acts of the computer-implemented method may be performed by a data processing system, which includes at least one data processing device. For example, the at least one data processing device is configured or adapted to perform the acts (e.g., steps) of the computer-implemented method. For this purpose, the at least one data processing device may, for example, store a computer program including instructions that, when executed by the at least one data processing device, cause the at least one data processing device to execute the computer-implemented method. The expressions “data processing system” and “at least one data processing device” may be used interchangeably, here and in the following. This holds also for respective expressions derived therefrom.

In case the at least one data processing device includes two or more data processing devices, certain acts carried out by the at least one data processing device may also be understood such that different data processing devices carry out different acts or different parts of an act. For example, it is not required that each data processing device carries out the acts completely. In other words, carrying out the acts may be distributed amongst the two or more data processing devices.

From each implementation of the computer-implemented method, a respective implementation of a method for denoising in X-ray imaging, which is not purely computer-implemented, is obtained by including respective acts of generating the X-ray image by the X-ray imaging system.

The X-ray image is, for example, a two-dimensional X-ray protection image that displays an object or a part of the object (e.g., a patient or a body part of the patient) that is, for example, located on a patient table of the X-ray imaging system. This object or the part of the object is denoted as image object in the following.

Each of the at least two noise level parameters is associated with one of the at least two frequency bands, which may be understood such that the total number of at least two noise level parameters is equal to the total number of the at least two frequency bands, and for each of the at least two frequency bands, a corresponding noise level parameter is received.

For example, the noise level parameter quantifies the noise content or noise level expected due to quantum noise and electronic noise in the associated frequency band. For example, the noise level parameter may correspond to the noise variance or noise standard deviation in the respective frequency band.

Since the X-ray image is a two dimensional image, the corresponding noise frequency spectrum is also a two-dimensional frequency spectrum. Consequently, a frequency band may, for example, be understood as a two-dimensional region (e.g., connected region) in the noise frequency domain. For example, a frequency band may be given by an area limited by two concentric circles in the noise frequency domain or, in other words, by a circular ring or annulus in the noise frequency domain.

The denoising algorithm may, for example, include a spatial denoising step and/or a temporal denoising step. The denoising algorithm (e.g., the spatial denoising step and/or the temporal denoising step) depends parametrically on the least two noise level parameters. Consequently, the denoising algorithm is more effective and consistent for different X-ray spectra involved in generating the respective X-ray image, and is more robust for varying X-ray spectra (e.g., during the course of fluoroscopy based interventions).

According to a number of (e.g., several) implementations, the denoising algorithm includes the application of a variance stabilizing transformation (e.g., a general Anscombe transformation, GAT), followed by the spatial denoising step and/or the temporal denoising step, followed by the application of the inverse of the variance stabilizing transformation.

Consequently, the denoising algorithm (e.g., the spatial denoising step and/or the temporal denoising step) achieves further improved results and, for example, more consistent results for different X-ray spectra involved in generating the respective X-ray image.

According to a number of (e.g., several) implementations, applying the denoising algorithm to the first X-ray image includes applying a trained machine learning model, MLM, to input data. The input data includes the first X-ray image or an image depending on the first X-ray image. The input data includes metadata that depends on or includes the at least two noise level parameters.

In general terms, a trained MLM may mimic cognitive functions that humans associate with other human minds. For example, by training based on training data, the MLM may be able to adapt to new circumstances and to detect and extrapolate patterns. Another term for a trained MLM is “trained function.”

In general, parameters of an MLM may be adapted or updated by training. For example, supervised training, semi-supervised training, unsupervised training, reinforcement learning, and/or active learning may be used. Further, representation learning, also denoted as feature learning, may be used. For example, the parameters of the MLMs may be adapted iteratively by a number of (e.g., several) steps of training. For example, within the training, a certain loss function, also denoted as cost function, may be minimized. For example, within the training of an artificial neural network, ANN, the backpropagation algorithm may be used.

For example, an MLM may include an ANN, a support vector machine, a decision tree, and/or a Bayesian network, and/or the MLM may be based on k-means clustering, Q-learning, genetic algorithms, and/or association rules. For example, an ANN may be or include a deep neural network, a convolutional neural network, or a convolutional deep neural network. Further, an ANN may be an adversarial network, a deep adversarial network, and/or a generative adversarial network.

According to a number of (e.g., several) implementations, the MLM is an ANN (e.g., a convolutional neural network, CNN, such as a U-Net).

Such MLMs have proven as particularly suitable image-to-image algorithms in the medical context.

The MLM is, for example, trained for denoising X-ray images (e.g., for spatial denoising). In other words, in such implementations, the denoising algorithm includes the spatial denoising step, and the spatial denoising step is implemented as the trained MLM.

Using the MLM for denoising is particularly beneficial in the framework of the present embodiments, since MLMs are particularly suitable to take into account additional information (e.g., the metadata in the present case) generically without knowing how exactly the additional information affects the denoising processes. This is due to the fact that the MLM has been specifically trained to take into account the metadata in an optimal manner.

The image depending on the first X-ray image on which the MLM is applied may, for example, correspond to a variance stabilized X-ray image generated by applying the variance stabilizing transformation to the first X-ray image. In some implementations, alternatively or in addition to the variance stabilizing transformation, the temporal denoising step may be applied to the first X-ray image before applying the MLM. However, in other implementations, the temporal denoising step may also be applied after applying the MLM (e.g., may be applied to the output of the MLM). In yet further implementations, the denoising algorithm does not contain the temporal denoising step.

The MLM may be a known MLM for X-ray denoising, which is adapted to be able to process the at least two noise level parameters as metadata. Therein, also, the training method for training the MLM may be known in principle and adapted to also include the metadata. For example, the algorithm described in the publication of S. Hariharan et al.: “Learning-based . . . ” may be used as a basis for the MLM.

According to a number of (e.g., several) implementations, the first X-ray image and the metadata are fused to generate fused input data, and the MLM is applied to the fused input data.

In other words, the first X-ray image and the metadata are fused at the input level of the MLM and processed jointly by the MLM. Consequently, the metadata is taken into account for generating the first denoised X-ray image in an efficient manner.

The fusion may be carried out in different ways. For example, the metadata may be written into a two-dimensional array and be treated analogously to an additional image channel. Instead of feeding only the first X-ray image (e.g., a two-dimensional single-channel image) into the MLM, a two-channel image having a first channel that corresponds to the first X-ray image and having a second channel that corresponds to the metadata is fed to the MLM. However, alternative fusion methods may also be used.

According to a number of (e.g., several) implementations, image features are generated by applying a first part of the MLM to the first X-ray image, and the image features and the metadata are fused to generate fused features. The first denoised X-ray image is generated by applying a second part of the MLM to the fused features.

In other words, the first X-ray image and the metadata are fused at an intermediate feature level of the MLM. Consequently, the metadata is taken into account for generating the first denoised X-ray image in an efficient manner. Further, the training of the MLM may be more efficient when the feature extraction is carried out at least partially based on the first X-ray image alone. Further, it may be possible to use an existing feature extraction module as the first part of the MLM without or with less adaptations to also take into account the metadata.

According to a number of (e.g., several) implementations, the first X-ray image corresponds to a first frame of a plurality of consecutive frames. A second X-ray image generated by the X-ray imaging system is received, where the second X-ray image corresponds to a second frame of the plurality of consecutive frames, which succeeds the first frame (e.g., succeeds the first frame directly or immediately). A difference image corresponding to a difference (e.g., a pixel-wise difference) between the first X-ray image and the second X-ray image is computed. A frequency decomposition is carried out to generate at least two respective variance maps of the difference image according to the at least two frequency bands. The denoising algorithm is applied to the first X-ray image depending on the at least two variance maps, and/or a second denoised X-ray image is generated by applying the denoising algorithm to the second X-ray image depending on the at least two variance maps.

The frequency decomposition may, for example, be carried out by transforming the difference image into the frequency domain (e.g., by using a fourier transform or a Laplacian transform or the method of a Laplacian pyramid, etc.). In the frequency domain, the transformed difference image may be separated into respective frequency maps corresponding to the at least two frequency bands, respectively. The frequency maps may then be back transformed into the image domain. The variance maps may then be generated by computing the respective variance of the back transformed frequency maps. Also, other approaches for the frequency decomposition are possible.

As a result, the at least two variance maps represent the variance of the difference between the first X-ray image and the second X-ray image in the at least two frequency bands. The denoising algorithm may then specifically take into account the different noise levels in the different frequency bands for the denoising, which leads to an improved quality of the denoising.

According to a number of (e.g., several) implementations, each image region of a plurality of image regions of the second X-ray image is classified as a static region or as a dynamic region depending on the at least two variance maps using the respective noise level parameter as a classification threshold. The denoising algorithm includes the temporal denoising step with an adjustable temporal denoising strength, and the temporal denoising strength is adjusted depending on a result of the classification for applying the temporal denoising step to the second X-ray image or to an image depending on the second X-ray image.

For example, commanding the denoising algorithm may also include the spatial denoising step, which may, for example, be carried out prior to the temporal denoising step or afterwards. For example, the image depending on the second X-ray image on which the temporal denoising step is applied may be generated by the spatial denoising step and, in some implementations, the variance stabilizing transformation.

The image regions may be connected groups of pixels (e.g., pixels within respective rectangular regions of the second X-ray image). However, the second X-ray image may also be partitioned in a different manner into the plurality of image regions.

A static image region may be understood as an image region having content that does not change or does not significantly change when comparing the first X-ray image to the second X-ray image. Analogously, a dynamic image region is an image region that is not a static image region. Consequently, adjusting the temporal denoising strength depending on the result of the classification allows to achieve an optimal trade-off between noise reduction and information loss due to the temporal denoising for each image region of the second X-ray image.

The temporal denoising step may, for example, be carried out by averaging over the corresponding image regions for two or more consecutive frames including the second frame. The averaging may also be a weighted averaging. The temporal denoising strength may, for example, be adjusted by adjusting the number of frames taken into account for the averaging and/or by adjusting the relative weights of the different frames.

Using the respective noise level parameter as a classification threshold may, for example, be understood such that the variance of the image region under consideration in the respective variance map is compared with the respective noise level parameter. If the variance is larger than twice the respective noise level parameter or larger than the noise level parameter plus a predefined tolerance, then the corresponding image region may, for example, be classified as dynamic and otherwise as static.

In some implementations, the temporal denoising step may analogously be applied to the first X-ray image.

According to a number of (e.g., several) implementations, if a number of image regions classified as static regions is larger than a predefined upper threshold value, the temporal denoising strength is adjusted to a predefined upper temporal denoising strength and the temporal denoising step is applied to all image regions of the plurality of image regions according to the upper temporal denoising strength.

In other words, in case the number of image regions classified as static regions is larger than the upper threshold value, then the whole second X-ray image may be considered as a static image and the same temporal denoising strength may uniformly be used for the whole second X-ray image. In this way, computational effort and time may be reduced.

In some implementations, the temporal denoising step may analogously be applied to the first X-ray image.

According to a number of (e.g., several) implementations, if the number of image regions classified as static regions is less than a predefined lower threshold value, which is less than the upper threshold value, the temporal denoising strength is adjusted to a predefined lower temporal denoising strength, which is smaller than the predefined upper temporal denoising strength. The temporal denoising step is applied to all image regions of the plurality of image regions according to the lower temporal denoising strength.

In other words, in case the number of image regions classified as static regions is less than the lower threshold value, then the whole second X-ray image may be considered as a dynamic image and the same temporal denoising strength may uniformly be used for the whole second X-ray image. In this way, computational effort and time may be reduced.

It is noted that the lower threshold value may also be zero in some implementations. Also, the lower temporal denoising strength may be zero in some implementations.

In some implementations, the temporal denoising step may analogously be applied to the first X-ray image.

According to a number of (e.g., several) implementations, if the number of image regions classified as static regions is less than the upper threshold value and larger than the lower threshold value, where the lower threshold value may be zero in some implementations, then the temporal denoising step is applied to all static image regions according to the upper temporal denoising strength; the temporal denoising step is applied to all dynamic image regions according to the lower temporal denoising strength, or the temporal denoising step is not applied to any of the dynamic image regions.

In other words, in case the second X-ray image is mixed in the sense that the second X-ray image both contains static image regions and dynamic image regions to some degree, the static image regions and the dynamic image regions are treated differently with different temporal denoising strengths.

Consequently, an optimal trade-off between noise reduction and information loss due to the temporal denoising may be achieved for the static image regions as well as for the dynamic image regions.

In some implementations, the temporal denoising step may analogously be applied to the first X-ray image.

According to a number of (e.g., several) implementations, the denoising algorithm includes the spatial denoising step with an adjustable spatial denoising strength. The spatial denoising step is applied to all dynamic image regions according to a predefined upper spatial denoising strength. The spatial denoising step is applied to all static image regions according to a predefined lower spatial denoising strength, which is less than the upper denoising strength. The upper spatial denoising strength and the lower spatial denoising strength are adjusted such that an overall denoising strength of the temporal denoising step and the spatial denoising step is constant or, in other words, is the same for all image regions of the plurality of image regions.

Consequently, the effectivity of the overall denoising algorithm is equally high for all image regions, while still the information lost due to the temporal denoising step is optimized. The overall denoising strength being constant may be understood such that variations of the overall denoising strength within a predefined tolerance range are allowed.

According to a number of (e.g., several) implementations, a set of imaging parameters of the X-ray imaging system corresponding to the generation of first X-ray image and, in respective implementations, the second X-ray image, is received. The at least two noise level parameters are determined depending on the set of imaging parameters (e.g., directly or indirectly).

The set of imaging parameters is given by a respective set of imaging parameters that have been used or were present or were applied when generating the respective X-ray image by the X-ray imaging system. The set of imaging parameters may include, for example, any parameters or conditions that affect or potentially affect the noise content (e.g., the quantum noise present in the X-ray image) and/or the X-ray spectrum. This may include exposure parameters, geometrical parameters, detector parameters of the X-ray detector, parameters of the imaged object, and so forth.

For example, denoting the variance of the total noise by σ²=σ_{q}²+σₙ², where σ_{q}² denotes the variance of the quantum noise and σₙ² denotes the variance of the electronic noise, the noise level function, NLF, may be denoted by σ²=α·yₘ+σₙ², with α=(σ²−σₙ²)/yₘ. Therein, yₘ corresponds to the mean signal in the resulting X-ray image. The parameter a depends on the X-ray spectrum and, consequently, on set of imaging parameters.

For example, α may also depend on the sensitivity of the pixels of the detector array. The sensitivity of the pixels may, for example, be taken into consideration via calibration images acquired at different X-ray spectra. Instead of a single α, it is also possible to use a map of α values: α[r, c] is then the value of a at the pixel location [r,c]. Analogously, a map for the electronic noise may also be used: σₙ²[r,c].

In some implementations, the parameter α may be determined based on the set of imaging parameters, and the at least two noise level parameters may be determined depending on the parameter α. In practice, for example, a database or lookup table relating the set of imaging parameters and/or the parameter α to the at least two noise level parameters may be provided. This is particularly beneficial to implement a real-time execution of the computer-implemented method.

According to a number of (e.g., several) implementations, the variance-stabilizing transformation is a generalized Anscombe transformation.

The generalized Anscombe transformation is a well-known variance-stabilizing transformation, which is also applicable in the context of X-ray images, and has proven particularly suitable for mixtures of Poisson and Gaussian noise. Consequently, a particularly reliable variance stabilization is achieved. The generalized Anscombe transformation is, for example, given by

$${y}^{′} = \frac{2}{α} \sqrt{α \cdot y + \frac{3}{8} {α}^{2} + {σ}_{n}^{2}} ,$$

where y denotes a pixel value of the X-ray image, y′ denotes the respective pixel value of the noise-variance-stabilized X-ray image, and

$${σ}_{n}^{2}$$

denotes the variance of the electronic noise of the X-ray detector, which is predetermined, for example, during a calibration phase. In case respective maps are used as mentioned above, this may be understood as

$${y}^{′} = \frac{2}{α [ r , c ]} \sqrt{α [ r , c ] \cdot y + \frac{3}{8} {α}^{2} [ r , c ] + {σ}_{n}^{2} [ r , c ]} ,$$

Alternatively, other variance-stabilizing transformations of the form

$${y}^{′} = f ( α ) \sqrt{h \cdot α + g ( α , {σ}_{n}^{2} )} ,$$

may be used, where f and g denote functions of α and

$$( α , {σ}_{n}^{2} ) ,$$

respectively, and h is a constant.

According to a number of (e.g., several) implementations, a plurality of variance stabilized reference X-ray images generated by the X-ray imaging system according to respective different sets of imaging parameters is received. An average reference image is generated by averaging the reference X-ray images. For each of the reference X-ray images of the plurality of variance stabilized reference X-ray images, a respective difference reference image corresponding to a difference (e.g., a pixel-wise difference) between the respective reference X-ray image and the average reference image is computed. For each of the difference reference images, a frequency decomposition is carried out according to the at least two frequency bands. For each of the at least two frequency bands, the respective noise level parameter of the at least two noise level parameter is computed depending on the noise variance in the respective frequency band of the difference reference images according to the frequency decomposition.

In some implementations, receiving the plurality of variance stabilized reference X-ray images includes receiving a plurality of reference X-ray images generated by the X-ray imaging system and applying the variance stabilizing transformation (e.g., the GAT) to each of the reference X-ray images.

The set of imaging parameters used for generating the respective X-ray image is different for different reference X-ray images in the sense that at least one imaging parameter of the set of imaging parameters differs. Therefore, each reference X-ray image has a different noise content and, consequently, leads to different noise level parameters in the at least two frequency bands.

According to a number of (e.g., several) implementations, the set of imaging parameters includes exposure parameters of the X-ray source (e.g., a peak kilovoltage of the X-ray source used for generating the X-ray image and/or a tube current of the X-ray source used for generating the X-ray image and/or an X-ray pulse duration used for generating the X-ray image).

Exposure parameters such as these are known to have a significant impact on the energy spectrum of the X-ray quanta emitted by the X-ray source. Consequently, these exposure parameters also affect the noise content in the X-ray image and, for example, the variance of the quantum noise and the noise level parameter significantly. Simulating the energy deposition depending on the exposure parameters therefore allows to determine the noise level parameter with increased accuracy and, consequently, makes the variance stabilization more effective.

According to a number of (e.g., several) implementations, the set of imaging parameters includes filter properties of an X-ray filter of the X-ray imaging system (e.g., a filter material and/or a filter thickness of the X-ray filter).

The X-ray filter is, for example, arranged between the X-ray source and the imaged object. The X-ray filter and the X-ray source may, for example, be part of a source unit of the X-ray imaging system (e.g., of a C-arm of the X-ray imaging system). The choice of the filter material may, for example, depend on an anode material of the X-ray source. A common choice for tungsten anodes is copper filters. However, other filter materials may include nickel, aluminum, or other metals.

The filter material as well as the filter thickness have a significant impact on the energy spectrum of the X-ray quanta after passing the X-ray filter. Consequently, they also affect the noise content in the X-ray image and, for example, the variance of the quantum noise and the noise level parameter significantly. Therefore, the at least two noise level parameters may be determined with increased accuracy.

According to a number of (e.g., several) implementations, the set of imaging parameters includes an X-ray dose at the X-ray detector.

The X-ray dose at the X-ray detector significantly affects the characteristics of noise at the detector. At very low dose levels, electronic noise may dominate the quantum noise, and hence, noise in different frequencies may differ. Therefore, the at least two noise level parameters may be determined with increased accuracy in such implementations.

According to a number of (e.g., several) implementations, the set of imaging parameters includes collimator properties of an X-ray collimator of the X-ray imaging system (e.g., a collimator opening size of the X-ray collimator).

The X-ray collimator is, for example, arranged between the X-ray filter and the imaged object. The X-ray collimator may, for example, be part of the source unit of the X-ray imaging system. The choice of the collimator opening size may, for example, depend on the part of the object, which shall be imaged.

The collimator opening size has a significant impact, for example, on the scattering of X-ray quanta at the collimator and, since the scattering is energy dependent, also on the energy spectrum of the X-ray quanta after passing the X-ray filter. Consequently, the collimator opening size also affects the noise content in the X-ray image and, for example, the variance of the quantum noise and the noise level parameters significantly. Therefore, the at least two noise level parameters may be determined with increased accuracy.

According to a number of (e.g., several) implementations, the set of imaging parameters includes a zoom factor used for generating the X-ray image.

The zoom factor corresponds to a part of a detector array of the X-ray detector, data of which is used for generating the X-ray image, and which may be smaller than the full detector array. While the zoom factor may have a smaller effect on the noise content of the X-ray image than the exposure parameters, the filter properties and the collimator properties, the effect may not be neglectable in some applications. Therefore, the at least two noise level parameters may be determined with increased accuracy.

According to a number of (e.g., several) implementations, the set of imaging parameters includes geometrical parameters of the X-ray imaging system and the imaged object, respectively.

The geometrical parameters may, for example, include a position and/or an orientation of the X-ray source with respect to the imaged object and/or a position and/or orientation of the X-ray detector with respect to the imaged object. For example, in case the X-ray imaging system includes a C-arm carrying the X-ray source and the X-ray detector, the geometrical parameters may include a position and/or an angulation (e.g., an angular position and an orbital position) of the C-arm.

The geometrical parameters may, for example, include, in some implementations, a position and/or an orientation of a patient table, on which the imaged object is located, or a part of the patient table.

The geometrical parameters may also include an effective thickness of the imaged object (e.g., in terms of a water equivalent thickness, WET).

The geometrical parameters have a significant impact, for example, on the scattering and transmission and, for example, absorption of X-ray quanta by the imaged object and, in some cases, further objects in the beam path. Consequently, the geometrical parameters also affect the noise content in the X-ray image and, for example, the variance of the quantum noise and the noise level parameters significantly. Therefore, the at least two noise level parameters may be determined with increased accuracy.

According to a number of (e.g., several) implementations, the set of imaging parameters includes a gain factor of the X-ray detector and/or a status parameter of an anti-scattering grid of the X-ray imaging system. The status parameter of the anti-scattering grid may, for example, be whether the anti-scattering grid is in the beam path or not.

The gain factor and the presence of the anti-scattering grid in the beam path have a significant impact, for example, on the noise content in the X-ray image and, for example, the variance of the quantum noise and the noise level parameters. Therefore, the at least two noise level parameters may be determined with increased accuracy.

According to a number of (e.g., several) implementations, the set of imaging parameters includes a pixel sensitivity of the detector pixels of the X-ray detector.

The sensitivity of the detector pixels may, for example, be taken into consideration via calibration images acquired at different X-ray spectra.

According to a further aspect of the present embodiments, an X-ray imaging method is provided. Therein, a first X-ray image is generated by an X-ray imaging system (e.g., by an X-ray source and an X-ray detector of the X-ray imaging system, such as controlled by a control system of the X-ray imaging system). A computer-implemented method for denoising in X-ray imaging according to the present embodiments is carried out based on the first X-ray image (e.g., by the control system and/or the data processing system), and the first denoised X-ray image or an image depending on the first denoised X-ray image (e.g., the back transformed denoised X-ray image) is displayed by a display device (e.g., a display device of the X-ray imaging system).

According to a number of (e.g., several) implementations, the X-ray imaging method is carried out as a fluoroscopy method, where a sequence of consecutive X-ray images including the first X-ray image is generated by the X-ray imaging system, and each of the consecutive X-ray images is denoised by using a computer-implemented method for denoising in X-ray imaging according to the present embodiments. Each denoised X-ray image or an image depending on the respective denoised X-ray image is, for example, displayed by the display device.

Further implementations of the X-ray imaging method according to the present embodiments follow directly from the various embodiments of the computer-implemented method for denoising in X-ray imaging according to the present embodiments, and vice versa. For example, individual features and corresponding explanations, as well as advantages relating to the various implementations of the computer-implemented method for denoising in X-ray imaging according to the present embodiments, may be transferred analogously to corresponding implementations of the X-ray imaging method according to the present embodiments.

According to a further aspect of the present embodiments, a computer-implemented training method for supervised training of a denoising algorithm (e.g., an MLM) for denoising in X-ray imaging is provided. Therein, at least two noise level parameters of an X-ray imaging system for at least two frequency bands are received, where each noise level parameter is associated with one of the at least two frequency bands. A loss function for the supervised training depends on the at least two noise level parameters.

For example, known methods for supervised training of an MLM (e.g., an ANN, such as CNN) may be used, where the original loss function is replaced by a corresponding loss function depending on the at least two noise level parameters. In this way, the MLM learns to optimally take into account at least two noise level parameters for denoising an X-ray image. The trained denoising algorithm will therefore be able to achieve a more effective denoising consistently for various X-ray spectra.

For example, the denoising algorithm (e.g., MLM) trained by the computer-implemented training method according to the present embodiments is an algorithm for spatial denoising and, for example, corresponds to the spatial denoising step mentioned above.

According to a number of (e.g., several) implementations, the loss function includes a weighted sum of errors according to the at least two frequency bands, where respective weighting factors of the weighted sum depend on or are given by the at least two noise level parameters.

In other words, the denoised X-ray image obtained during the training is compared to the corresponding ground truth individually for each of the at least two frequency bands to compute the errors for all of the at least two frequency bands. The loss function contains a corresponding term for each of the at least two frequency bands given by the respective error multiplied with the corresponding weighting factor for the respective noise frequency band. For example, in known training methods, only a single error for the whole noise frequency spectrum is considered. In some implementations, the same noise function may be used also in the computer-implemented training method, where the single error is replaced by the weighted sum of errors.

According to a number of (e.g., several) implementations of the computer-implemented method for denoising in X-ray imaging (e.g., such implementations where the trained MLM is used), the denoising algorithm (e.g., the MLM) is or has been trained by using a computer-implemented training method for supervised training according to the present embodiments.

According to a further aspect of the present embodiments, a data processing system that is configured to carry out a computer-implemented method for denoising in X-ray imaging according to the present embodiments is provided.

In the present disclosure, the expressions “data processing system” and “at least one data processing device” may be used interchangeably. A data processing device may, for example, be understood as a data processing device that includes processing circuitry. The data processing device can therefore, for example, process data to perform computing operations. This may also include operations to perform indexed accesses to a data structure (e.g., a look-up table, LUT), as well as a data processing process implemented in hardware.

For example, the data processing device may include one or more computers, one or more microcontrollers, and/or one or more integrated circuits (e.g., one or more application-specific integrated circuits, ASIC, one or more field-programmable gate arrays, FPGA, and/or one or more systems on a chip, SoC). The data processing device may also include one or more processors (e.g., one or more microprocessors, one or more central processing units, CPU, one or more graphics processing units, GPU, and/or one or more signal processors, such as one or more digital signal processors, DSP). The data processing device may also include a physical or a virtual cluster of computers or other of the units.

In various embodiments, the data processing device includes one or more hardware and/or software interfaces and/or one or more memory units.

A memory unit may be implemented as a volatile data memory (e.g., a dynamic random access memory, DRAM), or a static random access memory, SRAM, or as a non-volatile data memory (e.g., a read-only memory, ROM, a programmable read-only memory, PROM, an erasable programmable read-only memory, EPROM, an electrically erasable programmable read-only memory, EEPROM, a flash memory or flash EEPROM, a ferroelectric random access memory, FRAM, a magnetoresistive random access memory, MRAM, or a phase-change random access memory, PCRAM).

According to a further aspect of the present embodiments, a further data processing system that is configured to carry out a computer-implemented training method according to the present embodiments is provided.

According to a further aspect of the present embodiments, an X-ray imaging system is provided. The X-ray imaging system includes an X-ray source, an X-ray detector, and a control system that is configured to control the X-ray source and the X-ray detector to generate a first X-ray image. The X-ray imaging system further includes a data processing system that is configured to carry out a computer-implemented method for denoising in X-ray imaging according to the present embodiments.

The control system and the data processing system may be separated from each other. In this case, the control system may be understood to be or include a further data processing system according to the definition above. Alternatively, the data processing system of the X-ray imaging system includes the control system. For example, the control system may correspond to one or more data processing apparatuses of the data processing system.

For example, the X-ray imaging system includes a display device. The control system is configured to control the display device to display the denoised first X-ray image or an image depending on the denoised first X-ray image.

Further implementations of the X-ray imaging system according to the present embodiments follow directly from the various embodiments of the computer-implemented method, the computer-implemented training method, and the X-ray imaging method according to the present embodiments, and vice versa. For example, individual features and corresponding explanations as well as advantages relating to the various implementations of the computer-implemented method and the X-ray imaging method according to the present embodiments may be transferred analogously to corresponding implementations of the X-ray imaging system according to the present embodiments. For example, the X-ray imaging system according to the present embodiments is configured or programmed to carry out the X-ray imaging method according to the present embodiments. For example, the X-ray imaging system according to the present embodiments carries out the X-ray imaging method according to the present embodiments.

According to a further aspect of the present embodiments, a first computer program including first instructions is provided. When the first instructions are executed by a first data processing system, the instructions cause the first data processing system to carry out a computer-implemented method for denoising in X-ray imaging according to the present embodiments.

The first instructions may be provided as program code, for example. The program code may, for example, be provided as binary code or assembler and/or as source code of a programming language (e.g., C) and/or as program script (e.g., Python).

According to a further aspect of the present embodiments, a second computer program including second instructions is provided. When the second instructions are executed by an X-ray imaging system according to the present embodiments (e.g., by the data processing system of the X-ray imaging system and/or the control system of the X-ray imaging system), the second instructions cause the X-ray imaging system to carry out an X-ray imaging method according to the present embodiments.

The second instructions may be provided as program code, for example. The program code may, for example, be provided as binary code or assembler and/or as source code of a programming language (e.g., C) and/or as program script (e.g., Python).

According to a further aspect of the present embodiments, a third computer program including third instructions is provided. When the first instructions are executed by a third data processing system, the instructions cause the third data processing system to carry out a computer-implemented training method according to the present embodiments.

The third instructions may be provided as program code, for example. The program code may, for example, be provided as binary code or assembler and/or as source code of a programming language (e.g., C) and/or as program script (e.g., Python).

According to a further aspect of the present embodiments, a computer-readable storage medium storing a first computer program and/or a second computer program and/or a third computer program according to the present embodiments is provided.

The first computer program, the second computer program, the third computer program, and the computer-readable storage medium are respective computer program products including the first instructions and/or the second instructions and/or the third instructions.

Above and in the following, the solution according to the present embodiments is described with respect to methods and systems for denoising as well as with respect to methods and systems for providing a trained denoising algorithm. Features, advantages, or alternative embodiments herein may be assigned to the other claimed objects and vice versa. In other words, claims and embodiments for providing a trained denoising algorithm may be improved with features described or claimed in the context denoising in X-ray imaging. For example, datasets used in the methods and systems may have the same properties and features as the corresponding datasets used in the methods and systems for providing a trained denoising algorithm, and the trained denoising algorithms provided by the respective methods and systems may be used in the methods and systems denoising in X-ray imaging.

Further features and feature combinations of the present embodiments are obtained from the figures and their description as well as the claims. For example, further implementations of the present embodiments may not necessarily contain all features of one of the claims. Further implementations of the present embodiments may include features or combinations of features that are not recited in the claims.

In the following, the present embodiments will be explained in detail with reference to specific exemplary implementations and respective schematic drawings. In the drawings, same or functionally same elements may be denoted by the same reference signs. The description of same or functionally same elements is not necessarily repeated with respect to different figures.

### BRIEF DESCRIPTION OF THE DRAWINGS

FIG. 1 shows schematically an example implementation of an X-ray imaging system according to an embodiment;

FIG. 2 shows a schematic flow diagram of an example implementation of a computer-implemented method for denoising in X-ray imaging according to an embodiment;

FIG. 3 shows a schematic flow diagram of a further example implementation of a computer-implemented method for denoising in X-ray imaging according to an embodiment;

FIG. 4 shows examples of noise in the frequency domain for various bands;

FIG. 5 shows schematically an artificial neural network;

FIG. 6 shows schematically a convolutional neural network; and

FIG. 7 shows schematically a further convolutional neural network.

### DETAILED DESCRIPTION

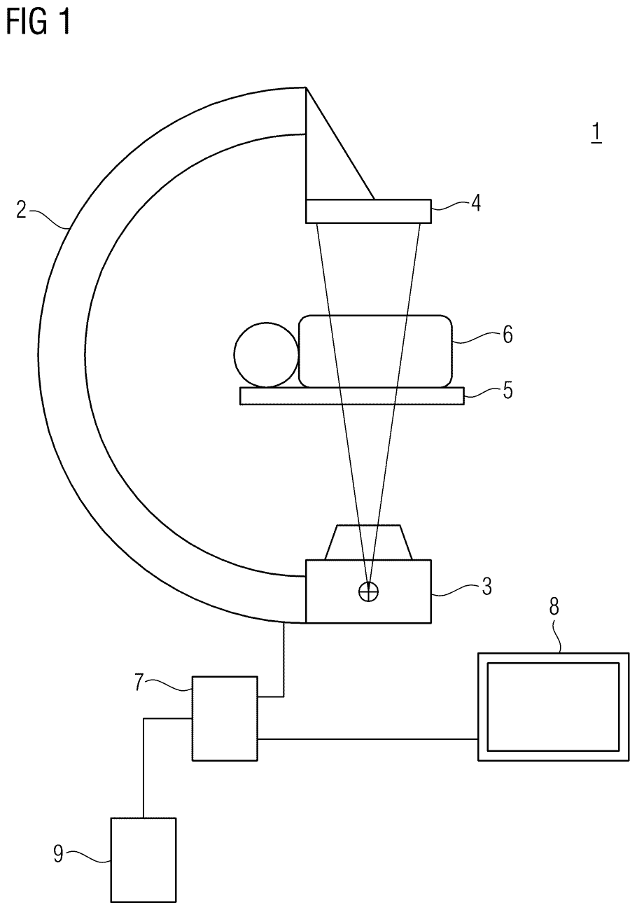

FIG. 1 shows schematically an example implementation of an X-ray imaging system 1 according to the present embodiments. The X-ray imaging system 1 includes a source unit 3 with an X-ray source, a detector unit 4 with an X-ray detector, and a control system 7 that is configured to control the X-ray source and the X-ray detector to generate X-ray images depicting an object 6 (e.g., a patient).

The X-ray imaging system 1 may, for example, include a patient table 5, on which the object 6 is arranged. The X-ray imaging system 1 includes a data processing system 9 according to the present embodiments. The data processing system 9 is configured to carry out a computer-implemented method for denoising in X-ray imaging according to the present embodiments. In the following, a number of (e.g., several) functions and method acts are described to be carried out by the control system 7, while other functions and method acts are described to be carried out by the data processing system 9. It is noted that the functions and method acts may also be distributed in different ways in alternative implementations.

For example, the control system 7 may adjust various imaging parameters of the X-ray imaging system 1 including, for example, exposure parameters such as a peak kilovoltage of the X-ray source, a tube current of the X-ray source, and/or an X-ray pulse duration. For example, the control system 7 may adjust further imaging parameters such as a filter material and/or filter thickness of an X-ray filter (e.g., a copper filter) by placing the appropriate X-ray filter into the beam path or by removing the appropriate X-ray filter from the beam path, respectively. For example, the control system 7 may adjust further imaging parameters such as a collimator opening size of an X-ray collimator. For example, the control system 7 may bring the X-ray collimator into the beam path or may remove the X-ray collimator from the beam path, respectively. For example, the control system 7 may adjust further imaging parameters such as a gain factor of the X-ray detector. For example, the control system 7 may bring an anti-scattering grid into the beam path or may remove the anti-scattering grid from the beam path, respectively.

In some implementations, the X-ray imaging system 1 includes a display device 8. The control system 7 is configured to control the display device 8 to display X-ray images or processed X-ray images. In case a fluoroscopy-based medical intervention is carried out, the X-ray images are, for example, generated as a sequence of consecutive frames to allow an operator to monitor or supervise the medical intervention. The X-ray imaging system 1 is, for example, configured to carry out an X-ray imaging method according to the present embodiments. An X-ray image is generated by the X-ray imaging system 1 using the X-ray source and the X-ray detector, and a computer-implemented method for denoising in X-ray imaging according to the present embodiments is carried out (e.g., by the data processing system 9). The denoised X-ray image is then, for example, displayed on the display device 8.

In some implementations, the X-ray imaging system 1 is configured as a fluoroscopy system (e.g., a C-arm fluoroscopy system). The source unit 3 and the detector unit 4 are attached opposite to each other on a C-arm 2 that may be rotated around different axes. The corresponding motions are denoted as angular and orbital motion, respectively. In some implementations, apart from the rotational motion of the C-arm 2, the patient table 5 and the C-arm 2 may be positioned relative to each other by respective translatory motions of the C-arm 2 and/or the patient table 5. Consequently, the position and/or orientation of the X-ray source with respect to the object 6 and the position and/or orientation of the X-ray detector with respect to the object 6 may be adjusted exactly to the desired imaging perspective.

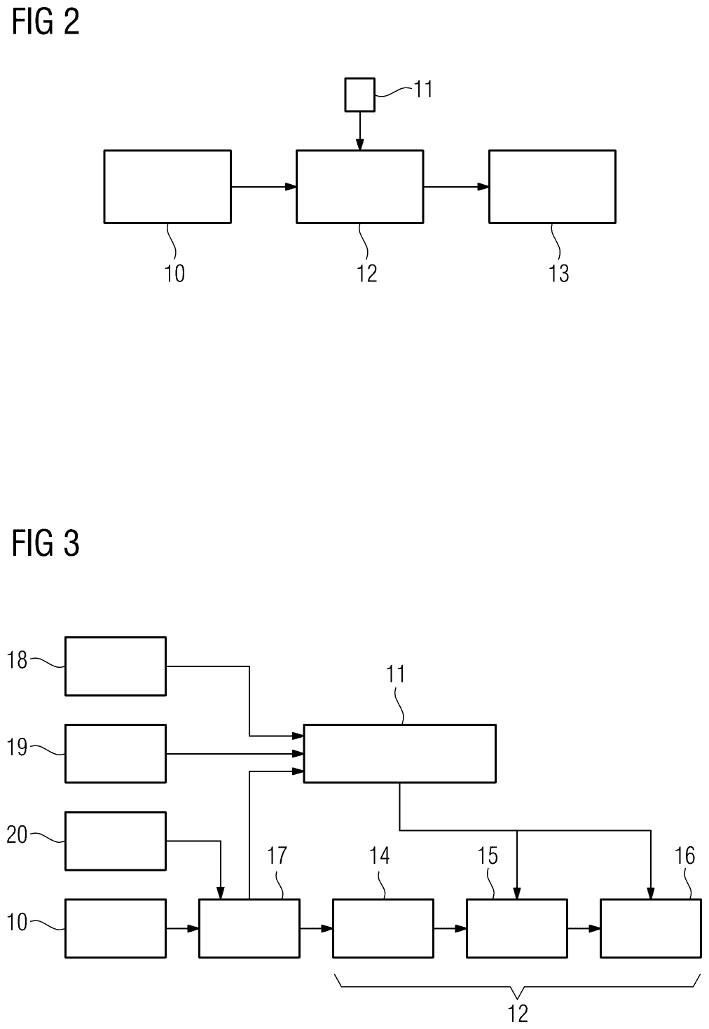

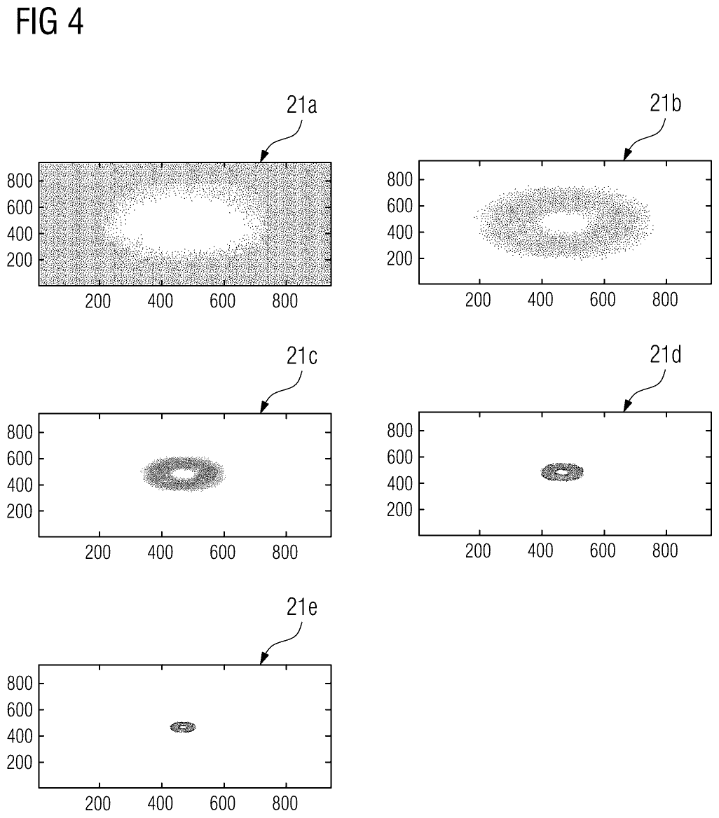

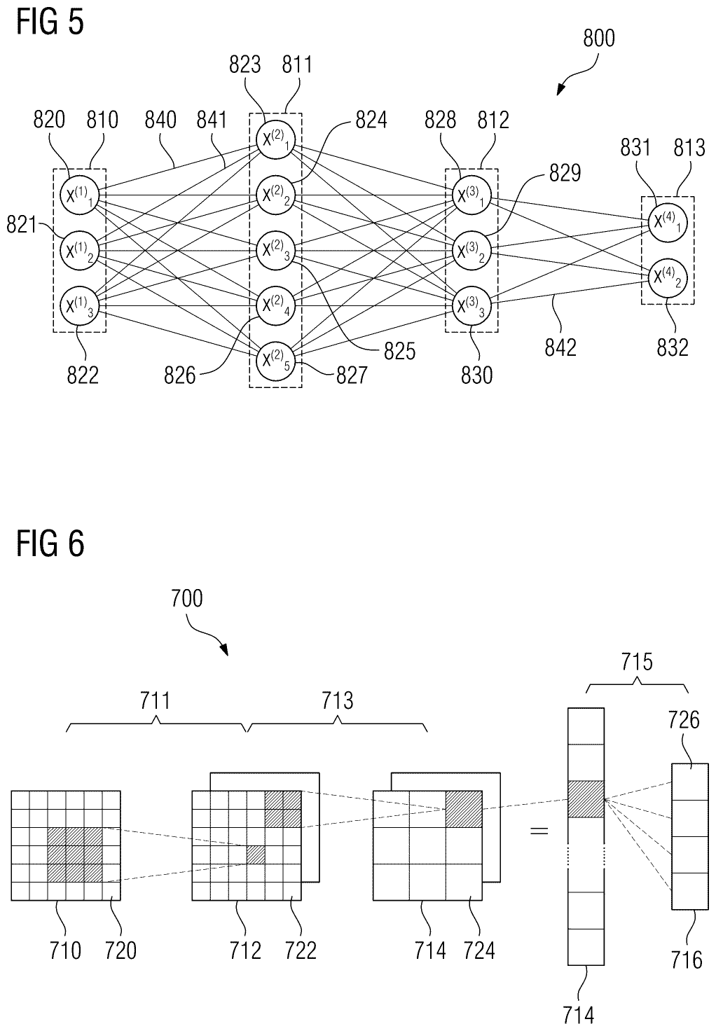

FIG. 2 shows a schematic flow diagram of an example implementation of a computer-implemented method for denoising in X-ray imaging according to the present embodiments. Therein, at least two noise level parameters 11 of the X-ray imaging system 1 for at least two frequency bands are received. Each noise level parameter 11 is associated with one of the at least two frequency bands. A first X-ray image 10 generated by the X-ray imaging system 1 is received. A first denoised X-ray image 13 is generated by applying a denoising algorithm 12 depending on the at least two noise level parameters 11 to the first X-ray image 10.

FIG. 3 shows a schematic flow diagram of a further example implementation of a computer-implemented method for denoising in X-ray imaging according to the present embodiments, which is based on the implementation of FIG. 2.

Therein, the NLF (e.g., the NLF parameters 17 α and σₑ² of the NLF given by σ²=α·yₘ+σₑ² with α=(σ²−σₑ²)/yₘ and yₘ) corresponding to the mean signal are determined. A variance-stabilized X-ray image 14 is generated for each X-ray image 10 of a plurality of consecutive X-ray images generated by the X-ray imaging system 1, for example, by applying a GAT

$${y}^{′} = \frac{2}{α} \sqrt{α \cdot y + \frac{3}{8} {α}^{2} + {σ}_{n}^{2}}$$

to each of the X-ray images 10.

Further, imaging parameters 18, 19, 20 of the imaging system 1 including, for example, exposure parameters 18, geometry parameters 19, and/or detector parameters 20 are received. The at least two noise level parameters 11 may, for example, be determined depending on the imaging parameters 18, 19, 20 and/or the NLF parameters 17. A spatial denoising step 15 is applied to each of the variance-stabilized X-ray image 14 followed by a temporal denoising step 16. The spatial denoising step 15 may, for example, be implemented by a trained MLM. The spatial denoising step 15 and/or the temporal denoising step 16 are applied depending on the and the NLF parameters 17. Applying the GAT may be considered as a part of the denoising algorithm 12. This is, however, not necessary. Further, an inverse GAT may be applied to the final output of the spatial denoising step 15 and the temporal denoising step 16.

For example, the noise level parameters 11 may correspond to the noise variance in the respective frequency band. As an example, the variance of noise in five different frequency bands for an X-ray image of a PMMA phantom with a water equivalent thickness, WET, of 30 cm acquired using 81 kVp and a 0.6 mm Cu prefiltration is shown in respective frequency domain images 21a, 21b, 21c, 21d, 21e.

In a number of (e.g., several) implementations of the present embodiments, a real-time capable hybrid spatio-temporal denoising approach that takes into consideration the noise characteristics associated with a given X-ray spectra in terms of the at least two noise level parameters 11 is provided.

In some implementations, a combination of learning-based classical denoising with temporal averaging based on the at least two noise level parameters 11 is provided.

The input X-ray spectrum, dose, and object thickness do in general change the noise in the different frequency bands. Existing strategies do not take into consideration such variations in the noise. This may result in a sub-optimal noise reduction. Therefore, in the present disclosure, different embodiments for denoising in different frequency bands are proposed, including but not limited to the following embodiments.

In some embodiments, the noise level function parameter a along with the effective object thickness (e.g., in terms of the WET), exposure parameters, and system geometry parameters define the noise level associated with different frequency bands. A high value for a may, for example, be associated with a hard beam. For example, a thick object absorbs low energy photons, and the remaining high energy photons contribute to image formation. Further, it is found that higher energy photons result in a higher α. In addition, α may also give details on the contribution from scattering.

This changes the amount of noise in different frequency bands. Therefore, in case the spatial denoising step 15 is implemented as a trained MLM, the at least two noise level parameters 11 may, for example, be used during the training and/or during the application of the MLM.

For example, during a supervised training of the MLM, the error in the different frequency bands may be weighted based on the at least two noise level parameters 11. These may also be stored as a look-up table and looked up when required. The at least two noise level parameters 11 may also be passed to the MLM as auxiliary metadata during training and inference.

In some embodiments, the noise level associated with the different frequency bands are used for identifying static and dynamic (e.g., moving) image regions in the acquired X-ray images 10. After applying the GAT, the variance of the difference between corresponding image regions of a pair of subsequent X-ray images 10 is analyzed in different frequency bands. In case the variance is close to twice the noise variance in the frequency band, the image region may be considered to be static.

For example, respective maps with the variance of differences in the frequency bands are first constructed. These maps are then analyzed to classify the image regions as static and dynamic regions. Strong temporal denoising may be applied for pixels associated with static regions. The maps may be spatially processed to provide smoothness.

In some embodiments, if most of the regions are identified to be static, which may be defined by a respective threshold setting, strong temporal denoising and weak spatial denoising is applied to the whole image. This is motivated by the reason that strong temporal denoising or even averaging results in an effective dose increase. For example, averaging two X-ray images is almost equivalent to doubling the dose, at least in certain situations.

In some embodiments, if there are static as well as dynamic regions to a relevant extent, which is defined by the threshold setting, strong temporal denoising may be applied in static regions and weaker temporal denoising or no temporal denoising at all may be applied in dynamic regions. Depending on the number of frames used for performing the temporal denoising, the amount of spatial denoising may be adapted to have the same amount of noise reduction across the image in total.

In some embodiments, in the case of spatial denoising based on the trained MLM, the amount of denoising may be controlled within the MLM. Alternatively, the MLM may be configured to remove noise to the maximum extent. Subsequently, a specified amount of the difference between the denoised and the input may be retained. The amount of difference introduced may vary spatially depending on the result of the analysis of the maps.

While known solutions do not account for varying noise levels in different frequency bands due to the X-ray spectrum at the X-ray detector, a number of embodiments use the signal-dependent noise variance along with details of the X-ray image acquisition (e.g., exposure parameters, geometry parameters, and so forth) to quantify the noise in difference frequency bands.

In some embodiments, this information is used during the development as well as the application of a machine learning-based denoising approach.

In some embodiments, the identified noise levels are, alternatively or additionally, used for efficiently guiding the spatio-temporal denoising approach by adaptively making the decisions on when and how to apply spatial denoising, temporal denoising or their combination.

The MLM may, for example, be an artificial neural network, ANN.

FIG. 5 displays an embodiment of an ANN 800 that is, for example, configured as an MLP. The ANN 800 includes nodes 820, . . . , 832 and edges 840, . . . , 842, where each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832. It is, however, also possible that the first node 820, . . . , 832 and the second node 820, . . . , 832 are the same. For example, in FIG. 5, the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as ingoing edge for the second node 820, . . . , 832 and as outgoing edge for the first node 820, . . . , 832.

In this example, the nodes 820, . . . , 832 of the artificial neural network 800 may be arranged in layers 810, . . . , 813, where the layers may include an intrinsic order introduced by the edges 840, . . . , 842 between the nodes 820, . . . , 832. For example, edges 840, . . . , 842 may exist only between neighboring layers of nodes. In the displayed example, there is an input layer 810 including only nodes 820, . . . , 822 without an incoming edge, an output layer 813 including only nodes 831, 832 without outgoing edges, and hidden layers 811, 812 in-between the input layer 810 and the output layer 813. In general, the number of hidden layers 811, 812 may be chosen arbitrarily. In an MLP, this number is at least one. The number of nodes 820, . . . , 822 within the input layer 810 may relate to the number of input values of the artificial neural network 800, and the number of nodes 831, 832 within the output layer 813 may relate to the number of output values of the artificial neural network 800.

For example, a real number may be assigned as a value to every node 820, . . . , 832 of the artificial neural network 800. Here, x(n); denotes the value of the i-th node 820, . . . , 832 of the n-th layer 810, . . . , 813. The values of the nodes 820, . . . , 822 of the input layer 810 are equivalent to the input values of the artificial neural network 800. The values of the nodes 831, 832 of the output layer 813 are equivalent to the output value of the artificial neural network 800. Further, each edge 840, . . . , 842 may include a weight being a real number. For example, the weight is a real number within the interval [−1, 1] or within the interval [0, 1]. Here, w^{(m,n)}ᵢⱼ denotes the weight of the edge between the i-th node 820, . . . , 832 of the m-th layer 810, . . . , 813 and the j-th node 820, . . . , 832 of the n-th layer 810, . . . , 813. Further, the abbreviation w^{(m,n)}_{i,j} is defined for the weight w^{(n,n+1)}_{i,j}. For example, to calculate the output values of the neural network 800, the input values are propagated through the neural network 800. For example, the values of the nodes 820, . . . , 832 of the (n+1)-th layer 810, . . . , 813 may be calculated based on the values of the nodes 820, . . . , 832 of the n-th layer 810, . . . , 813 by

$${x}_{j}^{( n + 1 )} = f ( {\sum }_{i} {x}_{i}^{( n )} {w}_{i , j}^{( n )} ) .$$

Herein, the function f is denoted as transfer function or activation function. Known transfer functions are step functions, the sigmoid functions (e.g., the logistic function, the generalized logistic function, the hyperbolic tangent, the arctangent function, the error function, the smoothstep function), or rectifier functions. The transfer function is, for example, used for normalization purposes. For example, the values are propagated layer-wise through the neural network 800. Values of the input layer 810 are given by the input of the neural network 800. Values of the first hidden layer 811 may be calculated based on the values of the input layer 810 of the neural network 800. Values of the second hidden layer 812 may be calculated based on the values of the first hidden layer 811, and so forth.

In order to set the values w^{(m,n)}_{i,j} for the edges, the neural network 800 is to be trained using training data. For example, training data includes training input data and training output data (e.g., denoted as tᵢ). For a training step, the neural network 800 is applied to the training input data to generate calculated output data. For example, the training data and the calculated output data include a number of values, the number being equal with the number of nodes of the output layer. For example, a comparison between the calculated output data and the training data is used to recursively adapt the weights within the neural network 800 (e.g., backpropagation algorithm). For example, the weights are changed according to

$${w}_{i , j}^{′ ( n )} = {w}_{i , j}^{( n )} - γ {δ}_{j}^{( n )} {x}_{i}^{( n )} ,$$

where γ is a predefined learning rate, and the numbers

$${δ}_{j}^{( n )}$$

may be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

based on

$${δ}_{j}^{( n + 1 )} ,$$

if the (n+1)-th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)-th layer is the output layer 813, where f′ is the first derivative of the activation function, and t⁽ⁿ⁺¹⁾ⱼ is the comparison training value for the j-th node of the output layer 813.

The MLM 19 may, for example, be a CNN. A convolutional neural network, CNN, is an ANN that uses a convolution operation instead of general matrix multiplication in at least one of its layers. These layers are denoted as convolutional layers. For example, a convolutional layer performs a dot product of one or more convolution kernels with the convolutional layer's input data, where the entries of the one or more convolution kernel are parameters or weights that may be adapted by training. For example, one may use the Frobenius inner product and the ReLU activation function. A convolutional neural network may include additional layers (e.g., pooling layers, fully connected layers, and/or normalization layers).

By using convolutional neural networks, the input may be processed in a very efficient way because a convolution operation based on different kernels may extract various image features so that by adapting the weights of the convolution kernel, the relevant image features may be found during training. Further, based on the weight-sharing in the convolutional kernels, fewer parameters need to be trained, which prevents overfitting in the training phase and allows to have faster training or more layers in the network, improving the performance of the network.

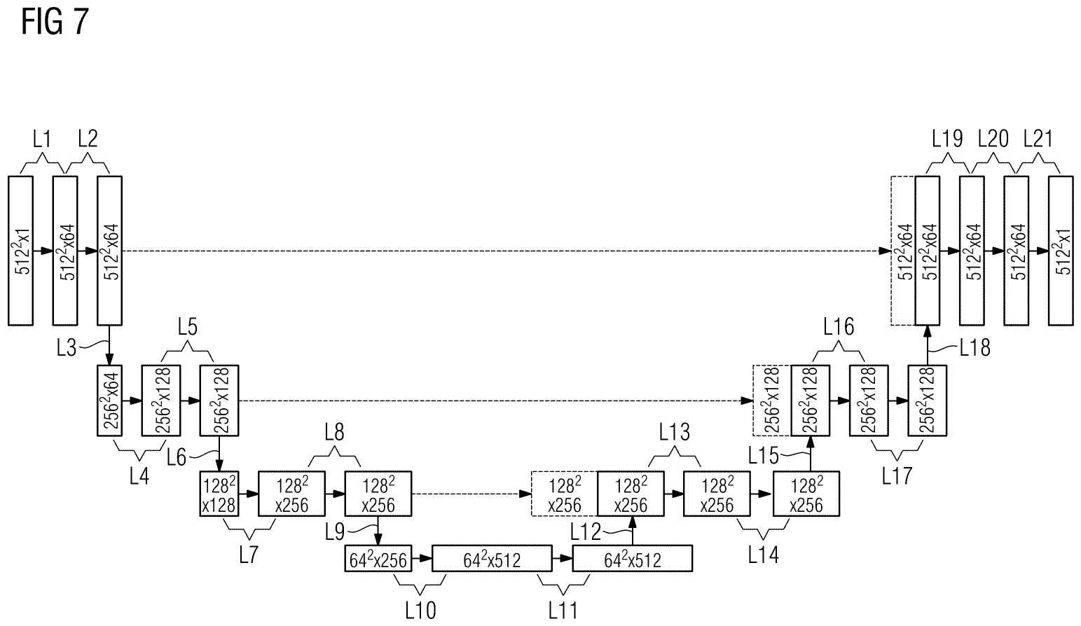

FIG. 7 displays an example embodiment of a convolutional neural network 700. In the displayed embodiment, the convolutional neural network 700 includes an input node layer 710, a convolutional layer 711, a pooling layer 713, a fully connected layer 714, and an output node layer 716, as well as hidden node layers 712, 714. Alternatively, the convolutional neural network 200 may include a number of (e.g., several) convolutional layers 711, a number of (e.g., several) pooling layers 713, and/or a number of (e.g., several) fully connected layers 715, as well as other types of layers. The order of the layers may be chosen arbitrarily; fully connected layers 715 may be used as the last layers before the output layer 716.

For example, within a convolutional neural network 700, nodes 720, 722, 724 of a node layer 710, 712, 714 may be considered to be arranged as a d-dimensional matrix or as a d-dimensional image. For example, in the two-dimensional case, the value of the node 720, 722, 724 indexed with i and j in the n-th node layer 710, 712, 714 may be denoted as x(n)[i, j]. However, the arrangement of the nodes 720, 722, 724 of one node layer 710, 712, 714 does not have an effect on the calculations executed within the convolutional neural network 700 as such, since these are given solely by the structure and the weights of the edges.

A convolutional layer 711 is a connection layer between an anterior node layer 710 with node values x(n−1) and a posterior node layer 712 with node values x(n). For example, a convolutional layer 711 is characterized by the structure and the weights of the incoming edges forming a convolution operation based on a certain number of kernels. For example, the structure and the weights of the edges of the convolutional layer 711 are chosen such that the values x(n) of the nodes 722 of the posterior node layer 712 are calculated as a convolution x(n)=K*x(n−1) based on the values x(n−1) of the nodes 720 anterior node layer 710, where the convolution * is defined in the two-dimensional case as

$${x}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Herein, the kernel K is a d-dimensional matrix (e.g., in the present example, a two-dimensional matrix) that may be small compared to the number of nodes 720, 722 (e.g., a 3×3 matrix or a 5×5 matrix). For example, this implies that the weights of the edges in the convolution layer 711 are not independent, but chosen such that the weights produce the convolution equation. For example, for a kernel being a 3×3 matrix, there are only 9 independent weights, each entry of the kernel matrix corresponding to one independent weight, irrespectively of the number of nodes 720, 722 in the anterior node layer 710 and the posterior node layer 712.

In general, convolutional neural networks 700 use node layers 710, 712, 714 with a plurality of channels (e.g., due to the use of a plurality of kernels in convolutional layers 711). In those cases, the node layers may be considered as (d+1)-dimensional matrices, the first dimension indexing the channels. The action of a convolutional layer 711 is then in a two-dimensional example defined as

$${x}_{b}^{( n )} [ i , j ] = {\sum }_{a} ( {K}_{a , b} * {x}_{a}^{( n - 1 )} [ i , j ] = {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ,$$

where

$${x}_{a}^{( n )}$$

corresponds to the a-th channel of the anterior node layer 710,

$${x}_{b}^{( n )}$$

corresponds to the b-th channel of the posterior node layer 712, and K_{a,b} corresponds to one of the kernels. If a convolutional layer 711 acts on an anterior node layer 710 with A channels and outputs a posterior node layer 712 with B channels, there are A·B independent d-dimensional kernels K_{a,b}.

In general, in convolutional neural networks 700, activation functions may be used. In this embodiment, rectified linear unit (ReLU) is used, with R(z)=max(0, z), so that the action of the convolutional layer 711 in the two-dimensional example is

$$\begin{matrix}{x}_{b}^{( n )} [ i , j ] = R ( {\sum }_{a} ( {K}_{a , b} * {x}_{a}^{( n - 1 )} [ i , j ] ) \\ = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ) .\end{matrix}$$

It is also possible to use other activation functions exponential linear unit (ELU), LeakyReLU, Sigmoid, Tanh, or Softmax.

In the displayed embodiment, the input layer 710 includes 36 nodes 720, arranged as a two-dimensional 6×6 matrix. The first hidden node layer 712 includes 72 nodes 722, arranged as two two-dimensional 6×6 matrices, each of the two matrices being the result of a convolution of the values of the input layer with a 3×3 kernel within the convolutional layer 711. Equivalently, the nodes 722 of the first hidden node layer 712 may be interpreted as arranged as a three-dimensional 2×6×6 matrix, where the first dimension corresponds to the channel dimension.

An advantage of using convolutional layers 711 is that spatially local correlation of the input data may be exploited by enforcing a local connectivity pattern between nodes of adjacent layers (e.g., by each node being connected to only a small region of the nodes of the preceding layer).

A pooling layer 713 is a connection layer between an anterior node layer 712 with node values x(n−1) and a posterior node layer 714 with node values x(n). For example, a pooling layer 713 may be characterized by the structure and the weights of the edges and the activation function forming a pooling operation based on a non-linear pooling function f. For example, in the two-dimensional case, the values x(n) of the nodes 724 of the posterior node layer 714 may be calculated based on the values x(n−1) of the nodes 722 of the anterior node layer 712 as

$${x}_{b}^{( n )} [ i , j ] = f ( {x}_{b}^{( n - 1 )} [ i {d}_{1} , j {d}_{2} ] , ... , {x}_{b}^{( n - 1 )} [ ( i + 1 ) {d}_{1} - 1 , ( j + 1 ) {d}_{2} - 1 ] ) .$$

In other words, by using a pooling layer 713, the number of nodes 722, 724 may be reduced by re-placing a number d1·d2 of neighboring nodes 722 in the anterior node layer 712 with a single node 722 in the posterior node layer 714 being calculated as a function of the values of the number of neighboring nodes. For example, the pooling function f may be the max-function, the average or the L2-Norm. For example, for a pooling layer 713, the weights of the incoming edges are fixed and are not modified by training.

The advantage of using a pooling layer 713 is that the number of nodes 722, 724 and the number of parameters is reduced. This leads to the amount of computation in the network being reduced and to a control of overfitting.

In the displayed embodiment, the pooling layer 713 is a max-pooling layer, replacing four neighboring nodes with only one node, the value being the maximum of the values of the four neighboring nodes. The max-pooling is applied to each d-dimensional matrix of the previous layer. In this embodiment, the max-pooling is applied to each of the two two-dimensional matrices, reducing the number of nodes from 72 to 18.

In general, the last layers of a convolutional neural network 700 may be fully connected layers 715. A fully connected layer 715 is a connection layer between an anterior node layer 714 and a posterior node layer 716. A fully connected layer 713 may be characterized by the fact that a majority (e.g., all) edges between nodes 714 of the anterior node layer 714 and the nodes 716 of the posterior node layer are present, and the weight of each of these edges may be adjusted individually.

In this embodiment, the nodes 724 of the anterior node layer 714 of the fully connected layer 715 are displayed both as two-dimensional matrices, and additionally as non-related nodes, indicated as a line of nodes, where the number of nodes was reduced for a better presentability. This operation is also denoted as flattening. In this embodiment, the number of nodes 726 in the posterior node layer 716 of the fully connected layer 715 is smaller than the number of nodes 724 in the anterior node layer 714. Alternatively, the number of nodes 726 may be equal or larger.

Further, in this embodiment, the Softmax activation function is used within the fully connected layer 715. By applying the Softmax function, the sum the values of all nodes 726 of the output layer 716 is 1, and all values of all nodes 726 of the output layer 716 are real numbers between 0 and 1. For example, if using the convolutional neural network 700 for categorizing input data, the values of the output layer 716 may be interpreted as the probability of the input data falling into one of the different categories.

For example, convolutional neural networks 700 may be trained based on the backpropagation algorithm. For preventing overfitting, methods of regularization may be used (e.g., dropout of nodes 720, . . . , 724, stochastic pooling, use of artificial data, weight decay based on the L1 or the L2 norm, or max norm constraints).

In the example of FIG. 7, the MLM is a CNN (e.g., a convolutional neural network having a U-Net structure). In the displayed example, the input data to the CNN is a two-dimensional medical image including 512×512 pixels, every pixel including one intensity value. The CNN includes convolutional layers indicated by solid, horizontal arrows, pooling layers indicating by solid arrows pointing down, and upsampling layers indicated by solid arrows pointing up. The number of the respective nodes is indicated within the boxes. Within the U-Net structure, first, the input images are downsampled (e.g., by decreasing the size of the images and increasing the number of channels). Afterwards, the images are upsampled (e.g., by increasing the size of the images and decreasing the number of channels) to generate a transformed image.

All except the last convolutional layers L1, L2, L4, L5, L.7, L8, L10, L11, L13, L14, L16, L17, L19, L20 use 3×3 kernels with a padding of 1, the ReLU activation function, and a number of filters or convolutional kernels that matches the number of channels of the respective node layers as indicated in FIG. 7. The last convolutional layer uses a 1×1 kernel with no padding and the ReLU activation function.

The pooling layers L3, L6, L9 are max-pooling layers, replacing four neighboring nodes with only one node, the value being the maximum of the values of the four neighboring nodes. The upsampling layers L12, L15, L18 are transposed convolution layers with 3×3 kernels and stride 2, which effectively quadruple the number of nodes. The dashed horizontal arrows correspond to concatenation operations, where the output of a convolutional layer L2, L5, L8 of the downsampling branch of the U-Net structure is used as additional inputs for a convolutional layer L13, L16, L19 of the upsampling branch of the U-Net structure. This additional input data is treated as additional channels in the input node layer for the convolutional layer L13, L16, L19 of the upsampling branch.

For training the CNN, a database of 500 first medical images was used, where the respective segmentation mask was created based on annotations of expert radiologists. For example, the experts determined for each of the 500 first medical images a segmentation mask for a structure of interest, where a value of 1 was assigned to pixels corresponding to the structure of interest, and a value of 0 was assigned to pixels not corresponding to the structure of interest. The database was split into training data (e.g., 320 datasets), validation data (e.g., 80 datasets), and test data (e.g., 100 datasets). For training the CNN, the backpropagation algorithm was used based on a binary cross-entropy cost function

$$L ( x , y ) = {\sum }_{i} {\sum }_{j} B C E ( y [ i , j ] , M ( x ) [ i , j ] )$$

$$with$$

$$B C E ( a , b ) := - a log ( b ) ( b ) - ( 1 - a ) log ( 1 - b ) ,$$

where x denotes a first medical image, y determines the corresponding segmentation mask created by the expert radiologist, and M(x) denotes the result of applying the CNN to the first input medical image x. Alternatively, one may use other cost functions such as weighted binary cross entropy, Focal Loss, or Dice Loss.

Based on the validation set of 80 datasets and the corresponding annotations, the best performing machine learning model out of a number of (e.g., several) machine learning models (e.g., with different hyperparameters, such as number of layers, size and number of kernels, padding, etc.) was selected. The specificity and the sensitivity were determined based on the test set including 100 datasets and the corresponding annotations.

Independent of the grammatical term usage, individuals with male, female or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for denoising in X-ray imaging, the computer-implemented method comprising:
receiving at least two noise level parameters of an X-ray imaging system for at least two frequency bands, wherein each noise level parameter of the at least two noise level parameters is associated with one of the at least two frequency bands;
receiving a first X-ray image generated by the X-ray imaging system;
generating a first denoised X-ray image, the generating of the first denoised X-ray image comprising applying a denoising algorithm depending on the at least two noise level parameters to the first X-ray image.

2. The computer-implemented method of claim 1, wherein applying the denoising algorithm to the first X-ray image comprises applying a trained machine learning model to input data comprising the first X-ray image and metadata,
wherein the metadata depends on the at least two noise level parameters.

3. The computer-implemented method of claim 2, further comprising:
fusing the first X-ray image and the metadata, such that fused input data is generated, and applying the machine learning model to the fused input data; or
generating image features, the generating of the image features comprising applying a first part of the machine learning model to the first X-ray image, and fusing the image features and the metadata, such that fused features are generated, wherein generating the first denoised X-ray image comprises applying a second part of the MLM to the fused features.

4. The computer-implemented method of claim 1, wherein the first X-ray image corresponds to a first frame of a plurality of consecutive frames, and
wherein the computer-implemented method further comprises:
receiving a second X-ray image generated by the X-ray imaging system, the second X-ray image corresponding to a second frame of the plurality of consecutive frames, the second frame succeeding the first frame;
computing a difference image corresponding to a difference between the first X-ray image and the second X-ray image;
carrying out a frequency decomposition, such that at least two respective variance maps of the difference image are generated according to the at least two frequency bands; and
applying the denoising algorithm to the first X-ray image depending on the at least two variance maps, generating a second denoised X-ray image, the generating of the second denoised X-ray image comprising applying the denoising algorithm to the second X-ray image depending on the at least two variance maps.

5. The computer-implemented method of claim 4, wherein:
each image region of a plurality of image regions of the second X-ray image is classified as a static region or as a dynamic region depending on the at least two variance maps using the respective noise level parameter as a classification threshold; and
the denoising algorithm comprises a temporal denoising step with an adjustable temporal denoising strength, and the temporal denoising strength is adjusted depending on a result of the classification for applying the temporal denoising step to the second X-ray image or to an image depending on the second X-ray image.

6. The computer-implemented method of claim 5, wherein:
when a number of image regions classified as static regions is larger than a predefined upper threshold value, the temporal denoising strength is adjusted to a predefined upper temporal denoising strength, and the temporal denoising step is applied to all image regions of the plurality of image regions according to the upper temporal denoising strength;
when the number of image regions classified as static regions is less than a predefined lower threshold value that is less than the upper threshold value, the temporal denoising strength is adjusted to a predefined lower temporal denoising strength that is smaller than the predefined upper temporal denoising strength, and the temporal denoising step is applied to all image regions of the plurality of image regions according to the lower temporal denoising strength; or
a combination thereof.

7. The computer-implemented method of claim 5, wherein when the number of image regions classified as static regions is less than the upper threshold value and larger than the lower threshold value:
the temporal denoising step is applied to all static image regions according to the upper temporal denoising strength; and
the temporal denoising step is applied to all dynamic image regions according to the lower temporal denoising strength, or the temporal denoising step is not applied to any of the dynamic image regions.

8. The computer-implemented method of claim 7, wherein the denoising algorithm comprises a spatial denoising step with an adjustable spatial denoising strength; and
wherein:
the spatial denoising step is applied to all dynamic image regions according to a predefined upper spatial denoising strength;
the spatial denoising step is applied to all static image regions according to a predefined lower spatial denoising strength that is less than the upper denoising strength; and
the upper spatial denoising strength and the lower spatial denoising strength are adjusted such that an overall denoising strength of the temporal denoising step and the spatial denoising step is constant for all image regions of the plurality of image regions.

9. The computer-implemented method of claim 1, further comprising:
receiving a set of imaging parameters of the X-ray imaging system corresponding to the generation of first X-ray image; and
determining the at least two noise level parameters depending on the set of imaging parameters.

10. The computer-implemented method of claim 9, further comprising:
receiving a plurality of variance stabilized reference X-ray images generated by the X-ray imaging system according to respective different sets of imaging parameters;
generating an average reference image, the generating of the average reference image comprising averaging the plurality of variance stabilized reference X-ray images;
for each variance stabilized reference X-ray image of the plurality of variance stabilized reference X-ray images, computing a respective difference reference image corresponding to a difference between the respective reference X-ray image and the average reference image;
for each of the difference reference images, carrying out a frequency decomposition according to the at least two frequency bands;
for each of the at least two frequency bands, computing the respective noise level parameter depending on the noise variance in the respective frequency band of the difference reference images.

11. The computer-implemented method of claim 9, wherein the set of imaging parameters comprises:
a peak kilovoltage of an X-ray source of the X-ray imaging system;
a tube current of the X-ray source of the X-ray imaging system;
an X-ray pulse duration;
a filter material, filter thickness, or the filter material and the filter thickness of an X-ray filter of the X-ray imaging system;
a collimator opening size of an X-ray collimator of the X-ray imaging system;
a position, orientation, or position and orientation of the X-ray source;
a position, orientation, or position and orientation of the X-ray detector;
a position, angulation, or position and angulation of a C-arm carrying the X-ray source and the X-ray detector;
a position, orientation, or position and orientation of a patient table or a part of the patient table;
an effective thickness of an imaged object;
a gain factor of the X-ray detector;
a status parameter of an anti-scattering grid of the X-ray imaging system;
a sensitivity of detector pixels of the X-ray detector; or
any combination thereof.

12. A computer-implemented training method for supervised training of a denoising algorithm for denoising in X-ray imaging, the computer-implemented training method comprising:
receiving at least two noise level parameters of an X-ray imaging system for at least two frequency bands, wherein each noise level parameter of the at least two noise level parameters is associated with one of the at least two frequency bands,
wherein a loss function for the supervised training depends on the at least two noise level parameters.

13. The computer-implemented training method of claim 12, wherein the loss function comprises a weighted sum of errors according to the at least two frequency bands, and
wherein respective weighting factors of the weighted sum depend on the at least two noise level parameters.

14. A data processing system comprising:
a processor configured to:
denoise in X-ray imaging, the processor being configured to denoise in X-ray imaging comprising the processor being configured to:
receive at least two noise level parameters of an X-ray imaging system for at least two frequency bands, wherein each noise level parameter of the at least two noise level parameters is associated with one of the at least two frequency bands;
receive a first X-ray image generated by the X-ray imaging system; and
generate a first denoised X-ray image, the generation of the first denoised X-ray image comprising application of a denoising algorithm depending on the at least two noise level parameters to the first X-ray image;
supervised train the denoising algorithm for denoising in X-ray imaging, the processor being configured to supervised train the denoising algorithm comprising the processor being configured to:
receive the at least two noise level parameters of the X-ray imaging system for the at least two frequency bands, wherein each noise level parameter of the at least two noise level parameters is associated with one of the at least two frequency bands, wherein a loss function for the supervised training depends on the at least two noise level parameters; or
a combination thereof.

15. An X-ray imaging system comprising:
an X-ray source;
an X-ray detector; and
a control system configured to control the X-ray source and the X-ray detector, such that a first X-ray image is generated;
a data processing system comprising:
a processor configured to:
denoise in X-ray imaging, the processor being configured to denoise in X-ray imaging comprising the processor being configured to:
receive at least two noise level parameters of the X-ray imaging system for at least two frequency bands, wherein each noise level parameter of the at least two noise level parameters is associated with one of the at least two frequency bands;
receive the first X-ray image generated by the X-ray imaging system; and
generate a first denoised X-ray image, the generation of the first denoised X-ray image comprising application of a denoising algorithm depending on the at least two noise level parameters to the first X-ray image;
supervised train the denoising algorithm for denoising in X-ray imaging, the processor being configured to supervised train the denoising algorithm comprising the processor being configured to:
receive the at least two noise level parameters of the X-ray imaging system for the at least two frequency bands, wherein each noise level parameter of the at least two noise level parameters is associated with one of the at least two frequency bands, wherein a loss function for the supervised training depends on the at least two noise level parameters; or
a combination thereof; and
a display device,
wherein the control system is further configured to control the display device to display the first denoised X-ray image.
